(19) United States
(12) Patent Application Publication
(10) Pub. No.: US 2026/0137273 A1
(43) Pub. Date: May 21, 2026

(54) FUNDUS IMAGING DEVICE

(71) Applicant: NIDEK CO., LTD., Gamagori (JP)

(72) Inventors: Shinya IWATA, Gamagori (JP); Masayuki YOSHINO, Gamagori (JP); Junpei NISHIYAMA, Gamagori (JP)

(73) Assignee: NIDEK CO., LTD., Gamagori (JP)

(21) Appl. No.: 19/336,901

(22) Filed: Sep. 23, 2025

(30) Foreign Application Priority Data
Sep. 30, 2024 (JP) 2024-171797

(51) Int. Cl. A61B 3/12; A61B 3/00

(52) U.S. Cl. CPC A61B 3/1225; A61B 3/0008

(57) ABSTRACT

A fundus imaging device has a fixation optical system, an OCT optical system, an imaging optical system, and a controller. The controller is configured to acquire a focus evaluation value based on a slit image formed by slit-shaped illumination light on a two-dimensional imaging element of the imaging optical system in a case where an optical scanner of the imaging optical system deflects in a predetermined direction. The controller is configured to perform diopter correction control of the OCT optical system by driving a first focus adjustment unit of the OCT optical system based on the focus evaluation value.

### CROSS-REFERENCE TO RELATED APPLICATIONS

This application is based on and claims priority under 35 USC 119 from Japanese Patent Application No. 2024-171797 filed on Sep. 30, 2024, the entire content of which is incorporated herein by reference.

### TECHNICAL FIELD

The present disclosure relates to a fundus imaging device.

### BACKGROUND ART

A fundus imaging device such as a fundus camera and an OCT device is widely used in the field of ophthalmology.

For example, a fundus imaging device described in JP2023-083084A includes an OCT optical system for obtaining a tomographic image of a fundus of a subject eye and a front imaging optical system for imaging a front image of the fundus. The front imaging optical system in the fundus imaging device described in JP2023-083084A is a slit scan type optical system.

In the fundus imaging device described in JP2023-083084A, an optical system that projects a focus adjustment index (hereinafter, referred to as a “focus index”) onto the fundus separately from illumination light is arranged in the front imaging optical system. The focus index described in JP2023-083084A is also referred to as a split index. The focus index is, for example, an index including two light flux separated by a deviation prism or the like, and is reflected in an observation image acquired via the front imaging optical system. The focus of the front imaging optical system is appropriately adjusted by changing a focus position of the front imaging optical system so that two index images in the observation image coincide with each other. In this case, the focus index is also used for focus adjustment of the OCT optical system by changing a focus position of the OCT optical system in conjunction with the front imaging optical system.

In addition, for example, a fundus imaging device described in JP2015-104581A includes a configuration for selectively turning on a plurality of fixation lamps (fixation targets) at different positions with respect to an optical axis in order to change an imaging range in a fundus.

During adjustment of the optical system such as focus adjustment, a fixation target is presented to a subject eye. In a case where the focus index is in a wavelength band visible to an examinee, both the fixation target and the focus index are formed in a point shape, making them easily confused, and thus the visual orientation of the examinee may be guided not to the fixation target but to the focus index. As a result, the adjustment of the optical system or the imaging may fail or be prolonged.

With respect to this, when the inventors of the present invention have studied forming the focus index with light in a wavelength band of an invisible region, it may be difficult to perform wavelength separation between the wavelength band of the focus index and the wavelength band of the measurement light in the OCT optical system. In this case, noise based on the focus index may occur when adjusting the OCT optical system.

### SUMMARY OF INVENTION

A technical object of the present disclosure is to provide a fundus imaging device capable of more reliably guiding a visual orientation of an examinee with respect to a fixation target during adjustment of an OCT optical system.

An aspect of the present disclosure relates to a fundus imaging device having:
a fixation optical system;
an OCT optical system;
an imaging optical system; and
a controller, in which
the fixation optical system is configured to present a fixation target on a fundus of a subject eye,
the OCT optical system includes:
   an OCT light source;
   a split optical element configured to split light emitted from the OCT light source into measurement light and reference light;
   a detector configured to detect a spectral interference signal between the measurement light reflected from the fundus of the subject eye and the reference light; and
   a first focus adjustment unit configured to adjust a focus position of the measurement light with respect to the fundus of the subject eye,
the imaging optical system includes:
   an irradiation optical system including a light source configured to emit infrared light as illumination light, and configured to form two light projection regions through which the illumination light passes on a pupil of the subject eye side by side in a first direction and irradiate the fundus of the subject eye with slit-shaped illumination light formed to be elongated along a second direction intersecting the first direction;
   an optical scanner configured to deflect the illumination light in the first direction on the fundus; and
   a light receiving optical system including a two-dimensional imaging element configured to form a light receiving region where fundus reflection light of the illumination light is extracted on the pupil of the subject eye so as to be sandwiched between the two light projection regions, and configured to receive the fundus reflection light of the illumination light, and
the controller is configured to:
   acquire a focus evaluation value based on a slit image formed by the slit-shaped illumination light on the two-dimensional imaging element of the imaging optical system in a case where the optical scanner deflects in a predetermined direction; and
   perform diopter correction control of the OCT optical system by driving the first focus adjustment unit of the OCT optical system based on the focus evaluation value.

According to the aspect of the present disclosure, it is possible to more reliably guide the visual orientation of the examinee with respect to the fixation target during adjustment of the OCT optical system.

### BRIEF DESCRIPTION OF DRAWINGS

Exemplary embodiment(s) of the present invention will be described in detail based on the following figures, wherein:

FIG. 1 is a diagram illustrating a schematic configuration of a fundus imaging device according to an example;

FIG. 2 is a diagram illustrating an outline of an optical system of the device according to the example;

FIG. 3 is a diagram illustrating shapes of light sources of an imaging optical system;

FIG. 4 is a diagram illustrating pupil division in the imaging optical system;

FIG. 5 is a diagram illustrating slit light emitted from the imaging optical system to a fundus of a subject eye;

FIG. 6 is a diagram illustrating an arrangement of fixation targets in a first fixation optical system;

FIG. 7 is a diagram illustrating an arrangement of fixation targets in a second fixation optical system;

FIG. 8A is a diagram illustrating fixation guidance and alignment in a case of imaging a peripheral portion of the fundus, and illustrates that fixation is guided by the fixation target presented by the second fixation optical system in a case where the subject eye is at a position farther than an appropriate operation distance;

FIG. 8B is a diagram illustrating the fixation guidance and the alignment in the case of imaging the peripheral portion of the fundus, and illustrates that fixation is guided by the fixation target presented by the first fixation optical system in a case where the subject eye reaches the appropriate operation distance;

FIG. 9 is a flowchart illustrating an operation flow of the device according to the example;

FIG. 10A is a diagram illustrating the slit light emitted from two light projection regions to the subject eye when a slit unit is arranged at a movement limit position on a minus diopter side;

FIG. 10B is a diagram illustrating the slit light emitted from the two light projection regions to the subject eye when diopter correction is appropriately performed on the subject eye;

FIG. 10C is a diagram illustrating the slit light emitted from the two light projection regions to the subject eye when the slit unit is arranged at a movement limit position on a plus diopter side;

FIG. 11 is a diagram illustrating a slit image formed on an imaging element, the slit image being corresponding to FIG. 10A; and

FIG. 12 is a diagram illustrating a slit image formed on an imaging element, the slit image being corresponding to FIG. 10B.

### DESCRIPTION OF EMBODIMENTS

### Overview

A fundus imaging device according to the present disclosure includes a fixation optical system, an OCT optical system, an imaging optical system, and a controller.

The fixation optical system is configured to present a fixation target on a fundus of a subject eye. The fixation target guides a visual orientation of the subject eye. The fixation target is projected as a fixation light flux of visible light. The fixation target may be projected at a position eccentric to optical axes of the OCT optical system and the imaging optical system. The OCT optical system includes an OCT light source, a split optical element configured to split light emitted from the OCT light source into measurement light and reference light, a detector configured to detect a spectral interference signal between the measurement light reflected from the fundus of the subject eye and the reference light, and a first focus adjustment unit configured to adjust a focus position of the measurement light with respect to the fundus of the subject eye. The imaging optical system includes an irradiation optical system, an optical scanner, and a light receiving optical system. The irradiation optical system includes a light source configured to emit infrared light as illumination light. The irradiation optical system is configured to form two light projection regions through which the illumination light passes on a pupil of the subject eye side by side in a first direction and irradiate the fundus of the subject eye with slit-shaped illumination light formed to be elongated along a second direction intersecting the first direction. The optical scanner is configured to deflect the illumination light in the first direction on the fundus. The light receiving optical system includes a two-dimensional imaging element configured to form a light receiving region where fundus reflection light of the illumination light is extracted on the pupil of the subject eye so as to be sandwiched between the two light projection regions, and configured to receive the fundus reflection light of the illumination light.

The controller is configured to acquire a focus evaluation value based on a slit image formed by the slit-shaped illumination light on the two-dimensional imaging element of the imaging optical system in a case where the optical scanner deflects in a predetermined direction. The controller is configured to perform diopter correction control of the OCT optical system by driving the first focus adjustment unit of the OCT optical system based on the focus evaluation value.

The focus evaluation value is acquired based on information correlated with a focus state in the slit image. For example, the focus evaluation value may be acquired based on position information of the slit image. However, the present invention is not necessarily limited thereto. Edge contrast, luminance information, or the like of the slit image can be used. When the edge contrast, the luminance information, or the like is used, the number of pixels exceeding a threshold may be adjusted to be minimized.

In the fundus imaging device according to the present disclosure, since a point-shaped focus index is not projected, it is possible to appropriately guide a visual orientation of the examinee with respect to the fixation target and image an OCT image. Even if the OCT image is acquired during focus adjustment, noise is less likely to occur in the OCT image.

The imaging optical system can be used as a fundus observation optical system. In this case, the controller may further acquire an observation image of the fundus of the subject eye based on a light receiving signal obtained from the two-dimensional imaging element in a case where the optical scanner performs scanning.

In the present disclosure, the imaging optical system may be capable of selectively emitting visible light for imaging and infrared light for observation as the illumination light. The visible light is used to image a color fundus image. The infrared light is used to image (acquire) a fundus observation image. In the focus adjustment, the controller may acquire the slit image by emitting infrared light as the slit-shaped illumination light. In this case, the visible light may be broadband light with a wavelength range of λ=400 nm to 750 nm. The infrared light may have a center wavelength within a range of λ=750 nm to 800 nm (more preferably, λ=770 nm to 790 nm).

In the present disclosure, the OCT optical system may be an SD-OCT optical system. In this case, the OCT light source may emit low-coherence light having a center wavelength included in a range of 820 nm to 880 nm. The detector may be a spectrometer. In general, in an SS-OCT optical system using measurement light having a center wavelength of about 1050 nm, a wavelength band between the measurement light and a visible region is wide, making it easy to project a split index in a wavelength band of an invisible region. However, in the SD-OCT optical system that emits the measurement light having the center wavelength of 820 nm to 880 nm, when the split index is projected in the wavelength band of the invisible region, wavelength separation is difficult, and thus it is particularly useful to perform focus adjustment based on the position information of the slit image formed by the slit-shaped illumination light.

In the present disclosure, it is not always necessary to continuously maintain the direction of the slit light in acquiring the focus evaluation value. In this case, for example, during focus adjustment, a direction of the optical scanner may be fixed to image the slit image at a time of acquiring the focus evaluation value, and scanning with the slit light may be performed to obtain the observation image for other periods except the time. Since the irradiation with the slit light is not continuously performed for a long time in a state where the direction is kept constant, fixation is less likely to be erroneously guided by the slit-shaped illumination light. In addition, while the fixation target is point-shaped and thus more likely to guide the visual orientation, the slit light is linear-shaped and is emitted to a relatively wider range, making it less likely to guide the visual orientation.

The fixation optical system may share a part of an optical system with the OCT optical system and the imaging optical system, and may present a peripheral fixation target for arranging an optic nerve head of the subject eye in a central portion of an imaging range. The fundus imaging device may further includes a fixation light source that is arranged around an objective lens in the OCT optical system and the imaging optical system, and is configured to present an external fixation target at a position corresponding to the peripheral fixation target. When guiding the visual orientation of the examinee so that the optic nerve head of the subject eye is arranged in the central portion of the imaging range, the external fixation target may guide the visual orientation of the examinee to a direction corresponding to the peripheral fixation target, from a stage where the subject eye is at a position farther than an appropriate operation distance. When the optical system is brought close to the subject eye within the operation distance while the subject eye is fixated on the external fixation target, it is desirable that the optical system is smoothly guided to the peripheral fixation target. Even if the slit light is in a visible wavelength band, it is easy to appropriately guide the visual orientation of the subject eye to the fixation target without being affected by the slit light.

### Example

Next, a fundus imaging device 1 according to an example will be described with reference to the drawings. The fundus imaging device 1 is a composite device of an OCT device and a slit scan type fundus imaging device.

The fundus imaging device 1 captures an OCT image of a subject eye E. Further, at least a color fundus image is imaged as a two-dimensional reflection image of a fundus Er.

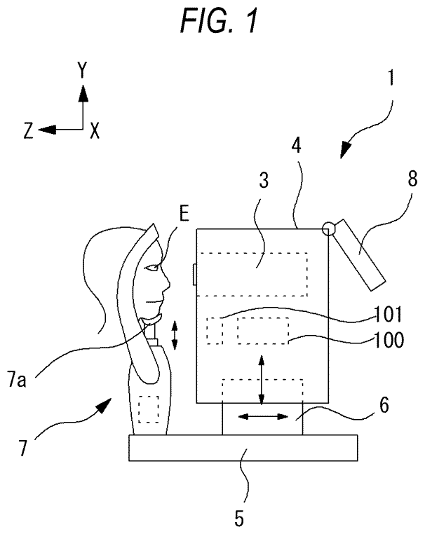

FIG. 1 is an external view of the fundus imaging device 1. In the present example, the fundus imaging device 1 includes an imaging unit 3, a base 5, a drive unit 6, a face support unit 7, a control unit 100, and a touch panel display 8 (hereinafter, referred to as “touch panel 8”). In the following description, for convenience, the unit in which the imaging unit 3 is installed is referred to as a device body. In the present example, the base 5, the drive unit 6, the face support unit 7, the control unit 100, and the touch panel 8 are a part of the device body. However, the control unit 100 and the touch panel 8 may be units separate from the device body. The base 5, the drive unit 6, and the face support unit 7 can be omitted as appropriate in the device body.

In the present example, the drive unit 6 moves the imaging unit 3 on the drive unit 6 in each of X, Y and Z directions with respect to the subject eye E. The drive unit 6 includes an actuator for moving the imaging unit 3 in each movable direction, and is driven based on a control signal transmitted from the control unit 100. The face support unit 7 supports the face of the subject. The face support unit 7 is fixed to the base 5. The face support unit 7 has a jaw support 7a. The jaw support 7a is movable in a vertical direction, thereby adjusting the height of the subject eye E according to an eye level of the device.

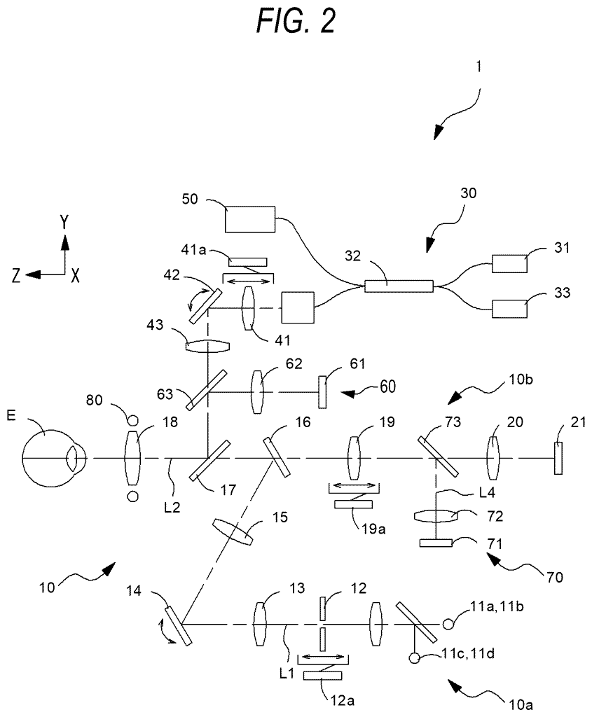

Next, FIG. 2 illustrates an optical system of the fundus imaging device 1. The fundus imaging device 1 includes at least a front imaging optical system 10, an OCT optical system 30, an anterior segment observation optical system 60, a first fixation optical system 70, and a second fixation optical system 80. As illustrated in FIG. 2, the front imaging optical system 10, the OCT optical system 30, and the first fixation optical system 70 share an objective optical system and are coaxial by a beam splitter/combiner (for example, a half mirror and a dichroic mirror).

### <OCT Optical System>

The OCT optical system 30 is a spectral domain OCT (SD-OCT) optical system. The OCT optical system 30 is used to image OCT data of the fundus Er. The OCT optical system 30 includes an OCT light source 31, a coupler (light splitter) 32, a measurement optical system 40, a reference optical system 50, and a detector 33.

In SD-OCT, a broadband light source is used as the OCT light source 31. As an example, the OCT light source 31 in the present example emits light having a center wavelength of λ=880 nm and a bandwidth of ±40 nm with respect to the center wavelength. However, the present invention is not necessarily limited thereto. For example, in a typical SD-OCT, light having a center wavelength of λ=800 nm to 900 nm is emitted from an OCT light source. The bandwidth may be, for example, about ±30 nm to ±60 nm with respect to the center wavelength. For example, the wavelength band of the light emitted from the OCT light source 31 may be changed in such a range.

The light emitted from the OCT light source 31 is split into measurement light (sample light) and reference light by the coupler 32. The measurement light is guided to the fundus Er via the measurement optical system 40. The reference light is guided to the reference optical system 50.

In the present example, the measurement optical system 40 includes a focusing lens 41, a focus lens 42, a scanning unit 43, a lens 44, and an objective lens 18.

The measurement light is guided to the scanning unit 43 via the focusing lens 41 and the focus lens 42. The scanning unit 43 performs two-dimensional scanning with the measurement light on the fundus Er. The scanning unit 43 is arranged at a position substantially conjugate with a pupil of the subject eye E. Thus, the measurement light is rotated about the pupil of the subject eye E. In the present example, for example, two galvanometer mirrors are used as the scanning unit 43. The fundus Er is irradiated with the measurement light passed through the scanning unit 43 via the objective lens 18. The measurement light reflected from the fundus Er is guided to the detector 33 after reversely tracing the measurement optical system 40.

In the present example, the reference optical system 50 is a reflective optical system and mainly includes a reference mirror (not illustrated). The reference light reciprocates once between the coupler 32 and the reference mirror. The reference light incident on the coupler 32 after one reciprocation is guided to the detector 33. The reference mirror is movable in an optical axis direction. An optical path length of the reference optical system 50 is changed according to a position of the reference mirror (not illustrated). As a result, an optical path length difference between the measurement light and the reference light is adjusted.

The present example shows a case where the reference optical system 50 is formed by the reflective optical system, but the reference optical system 50 may be formed by a transmissive optical system (for example, an optical fiber).

In the present example, a polarization state of the reference light is adjusted by a polarizer (not illustrated) arranged between the coupler 32 and the reference optical system 50. The polarizer may be arranged at a position where a polarization state of the measurement light is adjusted.

The detector 33 receives interference light caused by return light of the measurement light reflected from the fundus Er and the reference light. In SD-OCT, a spectrometer is used as the detector 33. The OCT data of the fundus Er is generated based on the spectral interference signal from the detector 33.

### <Front Imaging Optical System>

The front imaging optical system 10 acquires, as a captured image, a two-dimensional reflection image of the fundus based on the fundus reflection light. The front imaging optical system 10 is a slit scan type optical system. The front imaging optical system 10 scans the fundus of the subject eye with slit-shaped illumination light, and acquires a front image of the fundus based on reflection light from the fundus.

The front imaging optical system 10 includes an irradiation optical system 10a and a light receiving optical system 10b. The irradiation optical system 10a includes a light source unit 11, a slit unit 12, a lens 13, an optical scanner 14, a lens 15, a perforated mirror 16, a half mirror 17, the objective lens 18, and the like. The light receiving optical system 10b includes the objective lens 18, the half mirror 17, the perforated mirror 16, lenses 19 and 20, an imaging element 21, and the like.

The light source unit 11 includes a plurality of types of light sources having different wavelength bands. For example, the light source unit 11 includes visible light sources 11a and 11b and infrared light sources 11c and 11d. In the present example, the visible light emitted from the visible light sources 11a and 11b is used to image the color fundus image. The visible light may be, for example, white light. In the present example, the visible light sources 11a and 11b emit light in a band of λ=400 nm to 750 nm. However, the visible light sources 11a and 11b may emit a plurality of monochromatic visible light beams. For example, monochromatic light of three colors of red (R), green (G), and blue (B) may be emitted. In this case, the fundus may be irradiated with infrared (IR) light additionally or instead of the R light. However, the present invention is not limited thereto, and two colors of visible light may be combined, or five or more colors may be combined.

The infrared light emitted from the infrared light sources 11c and 11d is used to acquire (image) at least the fundus observation image. In the present example, the infrared light sources 11c and 11d emit light spreading in a band of λ=750 nm to 800 nm. However, the present invention is not necessarily limited thereto, and the wavelength band of the light from the infrared light sources 11c and 11d may be appropriately changed within a range on the longer wavelength side than the visible light emitted from the visible light sources 11a and 11b and on the shorter wavelength side than the visible light emitted from the visible light sources 11a and 11b. For example, the infrared light sources 11c and 11d may emit light having a center wavelength within a range of λ=750 nm to 800 nm (more preferably, λ=770 nm to 790 nm).

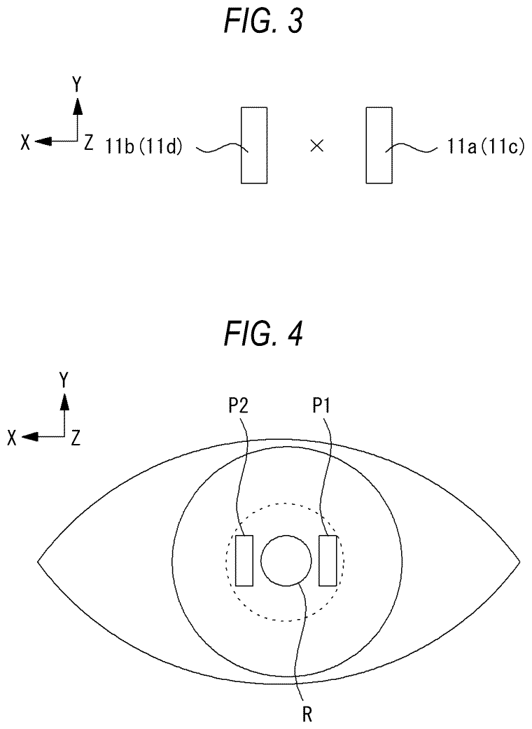

As described above, the light source unit 11 of the present example is provided with two light sources for each wavelength. The two light sources having the same wavelength are arranged away from an optical axis L on a pupil conjugate plane. The two light sources are arranged along the X direction, which is a scanning direction in FIG. 3, and are arranged axially symmetrically with respect to the optical axis L. As illustrated in FIG. 3, the outer peripheral shape of the two light sources may be a rectangular shape that is longer in a direction intersecting the scanning direction than in the scanning direction.

Light emitted from the two light sources passes through a lens and is applied to the slit unit 12. In the present example, the slit unit 12 has a slit formed to be elongated along the Y direction. Accordingly, the illumination light is formed in a slit shape on the fundus.

The optical scanner 14 scans the fundus with the illumination light. In the present example, scanning with the illumination light is performed in the X direction. As an example, the optical scanner 14 in the present example is a galvanometer scanner. However, the present invention is not necessarily limited thereto.

In the irradiation optical system 10a, the images of the two light sources are relayed by the optical system from the lens 13 to the objective lens 18 and formed on the pupil of the subject eye. That is, in the pupil of the subject eye, two pupil images on which light is incident toward the fundus are formed at positions separated in the scanning direction.

The illumination light is relayed by the optical system from the lens 13 to the objective lens 18 and forms an image on the fundus Er. Accordingly, the illumination light is formed in a slit shape on the fundus Er. The illumination light is reflected on the fundus Er and extracted from the pupil Ep.

The perforated mirror 16 is an optical path coupling unit that couples optical paths of the irradiation optical system 10a and the light receiving optical system 10b. The perforated mirror 16 reflects the illumination light emitted from the light source unit 11 toward the subject eye E, and allows a part of the fundus reflection light from the subject eye E that passes through the opening to pass toward the imaging element 21. Various beam splitters other than the perforated mirror 16 can be used as the optical path coupling unit.

Since the opening of the perforated mirror 16 is conjugate with the pupil of the subject eye, the fundus reflection light used for imaging is limited to a part that passes through an image of the perforated mirror opening (pupil image) on the pupil of the subject eye. Therefore, the image of the opening on the pupil of the subject eye is a light receiving region R in the present example. As illustrated in FIG. 4, the light receiving region R is formed between two light projection regions P1 and P2 (two light source images). As a result of appropriately setting the imaging magnification of each image, the diameter of the opening, and the arrangement interval of the two light sources, the light receiving region R and the two light projection regions P1 and P2 are formed so as not to overlap each other on the pupil.

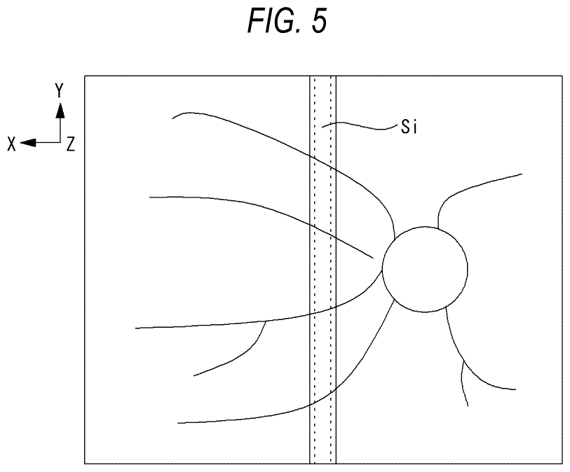

The fundus reflection light that passes through the objective lens 18 and the opening of the perforated mirror 16 forms a slit-shaped image Si at a fundus conjugate position via lenses 25a and 25b (see FIG. 5).

The imaging element 21 is arranged at the fundus conjugate position. The imaging element 21 has sensitivity to both infrared light and visible light. In the present example, a CMOS having a two-dimensional light receiving surface is used as the imaging element 21. The image Sr of the slit-shaped region of the fundus Er is projected onto the imaging element 21. In the present example, as the fundus Er is scanned with the slit-shaped illumination light, an image (slit-shaped image Sr) at a scanning position on the fundus Er is sequentially projected. In this way, the entire image of the scanning range is projected onto the imaging element 21 in a time-division manner. As a result, a front image (two-dimensional reflection image) of the fundus is imaged as the entire image of the scanning range.

Here, in the present example, harmful light is removed by displacing a region exposed on an imaging surface in synchronization with the scanning unit in the irradiation optical system 10a by a rolling shutter function of the CMOS. Instead of the rolling shutter function, a liquid crystal shutter or the like may be used as a scanning unit that electronically scans the slit. In this case, an imaging element that performs exposure and readout with a global shutter can be adopted. The width of the region exposed on the imaging surface is desirably wider than a width corresponding to the slit light projected onto the fundus.

The front imaging optical system 10 includes a diopter correction unit. In the present example, focus adjustment is performed in each of the irradiation optical system 10a and the light receiving optical system 10b. A focus adjustment unit in the irradiation optical system 10a includes the slit unit 12 and a drive unit 12a. The drive unit 12a changes the position of the slit unit 12 along an optical axis L1. The focus adjustment is appropriately performed by adjusting the position of the slit unit 12 such that an opening of the slit unit 12 is conjugate with the fundus. A focus adjustment unit in the light receiving optical system 10b includes the lens 19 and a drive unit 19a. The drive unit 19a changes the position of the lens 19 along an optical axis L2. The focus adjustment is appropriately performed by adjusting the position of the lens 19 such that the imaging element 21 is conjugate with the fundus. The slit unit 12 and the lens 19 are driven in conjunction with each other. In the following description, in describing the positions of the slit unit 12 and the lens 19, a position corresponding to the 0D eye of each member is used as a reference, a position corresponding to myopia is referred to as a position on a minus diopter side, and a position corresponding to hyperopia is referred to as a position on a plus diopter side.

### <Anterior Segment Observation Optical System>

The anterior segment observation optical system 60 images the anterior segment of the subject eye E to acquire an anterior segment observation image. In the present example, the anterior segment observation optical system 60 illuminates the anterior segment with infrared light as observation light from an infrared light source (not illustrated). An imaging element 61 receives reflection light from the anterior segment. A front image of the anterior segment is acquired as the anterior segment observation image based on a signal transmitted from the imaging element 61. The observation image of the anterior segment is used for alignment and tracking control of the imaging unit 3 with respect to the subject eye E during fundus imaging.

### <First Fixation Optical System>

The first fixation optical system 70 is provided to project a fixation target onto the fundus of the subject eye. The visual orientation of the examinee is guided with respect to the fixation target. In the present example, the first fixation optical system 70 can change the presentation position of the fixation target.

The first fixation optical system 70 includes, for example, a fixation target unit 71 and a relay lens 72. The first fixation optical system 70 shares an optical path from a dichroic mirror 73 to the objective lens 18 with the front imaging optical system 10. An optical axis L3 passing through the center of the fixation target unit 71 is made coaxial with the optical axis L2 by the dichroic mirror 73.

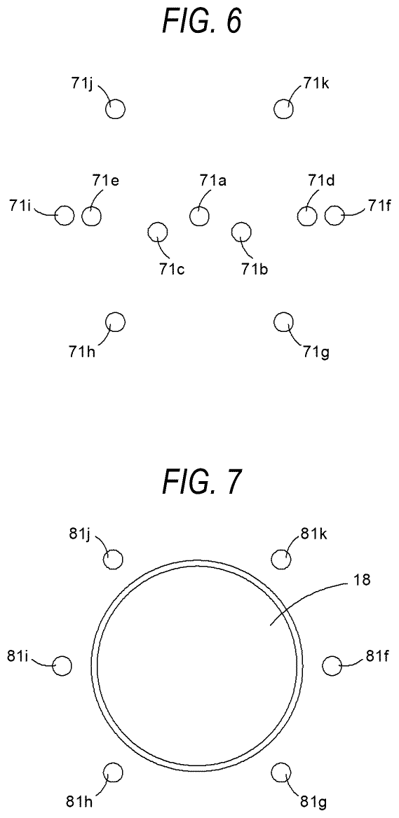

FIG. 6 illustrates the fixation target unit 71 viewed from the front. The fixation target unit 71 includes a plurality of fixation light sources 71a to 71k that emit visible light. For example, LEDs may be used as the fixation light sources 71a to 71k. The presentation position of the fixation target is determined in advance, and the fixation light sources 71a to 71k are arranged at positions corresponding to the respective presentation positions. In the present example, the presentation position of the fixation target is changed by selectively turning on any one of the fixation light sources 71a to 71k. However, the configuration of the fixation target unit 71 is not necessarily limited thereto, and may be a dot matrix display body (for example, a dot matrix LED) in which fixation light sources are two-dimensionally arranged on a substrate, or may be a liquid crystal display. Alternatively, the fixation light flux may be deflected by an optical scanner to present the fixation target at any position in a stepless manner.

In the fixation target unit 71, the fixation light source 71a is arranged on the optical axis L2, and the fixation light sources 71b to 71k are arranged at positions away from the optical axis L2. The fixation light source 71a arranged on the optical axis L2 is used, for example, when the macula is set as a center of an imaging range. The fixation light sources 71b and 71c are used when the middle between the macula and the optic nerve head are set as the center of the imaging range. The other fixation light sources 71d to 71k are used to image the peripheral portion of the fundus. For example, the fixation light sources 71d and 71e are used when the optic nerve head is set as the center of the imaging range. The fixation light sources 71b and 71d are used to image the right eye, and the fixation light sources 71c and 71e are used to image the left eye. The fixation light sources 71f to 71k are used for panoramic imaging or the like.

### <Second Fixation Optical System>

When imaging the peripheral portion of the fundus, the second fixation optical system 80 is used to guide the visual orientation of the examinee, from a stage where the subject eye E is at a position farther than an appropriate operation distance WD2 with respect to the imaging unit 3. FIG. 7 illustrates the second fixation optical system 80 viewed from the front. As illustrated in FIG. 7, the second fixation optical system 80 includes at least a plurality of fixation light sources 81f to 81k. The fixation light sources 81f to 81k are arranged around the objective lens 18 at positions corresponding to the fixation light sources 71d to 71k for peripheral imaging in the fixation target unit 71.

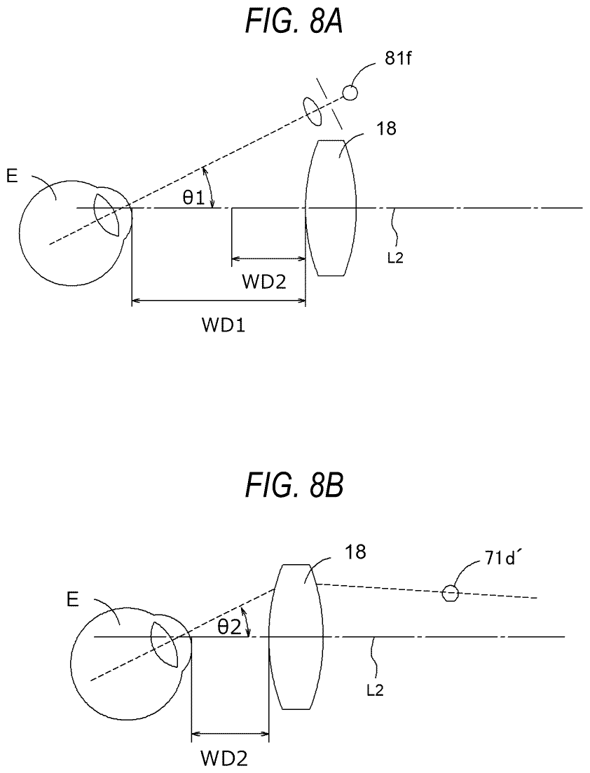

As illustrated in FIG. 8A, when the visual orientation of the subject eye is guided to the fixation target formed by the fixation light source 71d or 71f of the fixation target unit 71, the fixation light source 81f of the second fixation optical system 80 is turned on. The fixation light source 81f can be visually recognized as inclining the visual orientation by θ1 with respect to the optical axis L2 at a stage where an operation distance is WD1 larger than the appropriate value WD2. By moving the imaging unit 3 closer to the subject eye E, as illustrated in FIG. 8B, the visual orientation of the subject eye is smoothly guided with respect to the fixation target formed by the fixation light source 71d or 71f of the fixation target unit 71 at the stage where the operation distance becomes the appropriate value WD2. It is desirable that the inclination θ2 of the visual orientation with respect to the fixation target formed by the fixation light source 81f substantially coincides with θ1.

### <Control Unit>

The control unit 100 is a processing device (processor) that performs control processing of each unit and arithmetic processing. The control unit 100 includes a CPU, a RAM, a ROM, and the like. For convenience, the control unit 100 performs image processing on various images obtained by the fundus imaging device 1. In other words, the control unit 100 also serves as an image processing unit.

The control unit 100 is electrically connected to the OCT optical system 30, the drive unit 12a, the drive unit 19a, a drive unit 41a, the front imaging optical system 10, the anterior segment observation optical system 60, a storage unit 101, the touch panel 8, and the like.

The storage unit 101 may be a non-transitory storage medium capable of storing storage contents even when a supply of power is cut off. For example, the storage unit 101 stores various control programs, fixed data, and the like. In addition, for example, the storage unit 101 stores a captured image obtained by the fundus imaging device 1. The captured image may be transferred to an external storage device (for example, a storage device connected to the control unit 100 via a LAN and a WAN).

The storage unit 101 stores an imaging control program. Operations described below are executed according to the imaging control program.

### <Description of Operation>

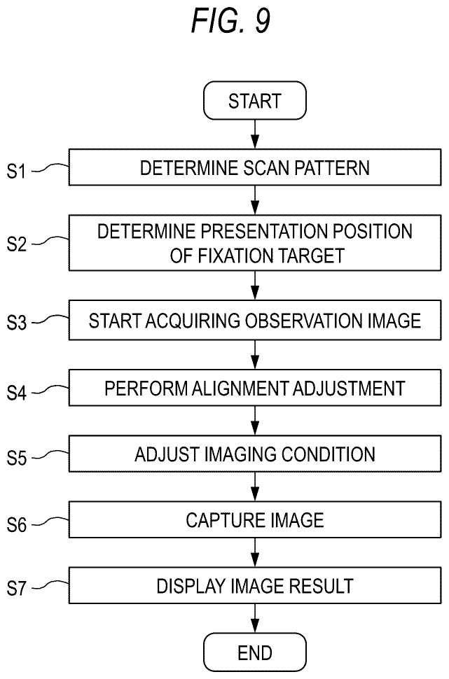

Next, the operation of the fundus imaging device 1 according to the present example will be described with reference to the flowchart illustrated FIG. 9. For convenience, in the following description, a case will be described in which the OCT data of the fundus is imaged in one type of scan pattern in the fundus imaging device 1.

In an examination, an examiner causes the face support unit 7 to support the face of the subject in advance. First, the control unit 100 determines the scan pattern and the presentation position of the fixation target (steps S1, S2). Examples of the type of the scan pattern include line scan, cross scan, multi-scan, radial scan, and raster scan (also referred to as map scan). In the present example, the presentation position of the fixation target is used as a reference (center) of the scan pattern on the fundus. In the present example, the presentation position of the fixation target can be selected from the positions corresponding to the fovea, the optic nerve head, and the fundus center (the middle between the fovea and the optic nerve head). The combination of the scan pattern and the presentation position of the fixation target is selected and further determined based on, for example, an operation input by the examiner. At this stage, one of the right eye, the left eye, and both eyes is selected as the imaging target eye.

When the scan pattern and the presentation position of the fixation target are determined, the control unit 100 starts acquisition of an observation image (step S3). As the observation image, acquisition of an anterior segment observation image and a fundus observation image are started. In addition, the OCT optical system 30 is controlled to start acquisition of an OCT image for adjustment. As a typical example of the OCT image for adjustment, in the present example, an image of an XZ cross section passing through the optical axis L is acquired. The OCT image for adjustment is repeatedly acquired. In addition, the fixation light source in the first fixation optical system 70 is started to be turned on according to the determined presentation position. When the fixation light source 71d or 71e corresponding to the optic nerve head is selected, the corresponding fixation light source 81f or 81j in the second fixation optical system 80 is additionally turned on.

Next, alignment adjustment is performed (step S4). The control unit 100 adjusts the position of the imaging unit 3 with respect to the subject eye E to a position where the subject eye E can be imaged based on the observation image. In the present example, the position of the imaging unit 3 is adjusted from a predetermined initial position to a state where the optical axis of the imaging unit 3 coincides with the subject eye E and the appropriate operation distance is obtained. The position of the imaging unit 3 is automatically moved with respect to the subject eye E based on the anterior segment observation image. In the present example, after an alignment index (not illustrated) is projected and the positional relation in each of the XYZ directions is adjusted, the imaging optical axis is adjusted to coincide with the pupil center in the XY direction.

### <Adjustment of Imaging Conditions>

After the alignment is completed, the control unit 100 performs adjustment processing of the OCT optical system 30 and the front imaging optical system 10 (step S5). In the present example, as an example, each of the OPL, the focus, and the polarization in the OCT optical system 30 and the focus of the front imaging optical system 10 are adjusted to at least a predetermined state.

### <Focus Adjustment>

Here, the focus adjustment will be described in detail.

First, the control unit 100 moves the slit unit 12 and the focusing lens 19 in the front imaging optical system 10 and the focusing lens 41 in the OCT optical system 30 to initial positions. In the present example, the initial position is a movement limit position on the minus diopter side. However, the initial position is not necessarily limited thereto, and may be a movement limit position on the plus diopter side, or may be an intermediate position (for example, a position corresponding to 0D) between the movement limit positions. The direction of deflection of the slit light by the optical scanner 14 of the front imaging optical system 10 is fixed to a predetermined direction. As an example, the direction is fixed to 0°. In this case, the centers of the two slit light guided from the optical scanner 14 to the subject eye E coincide with the optical axis L2.

The control unit 100 turns on the infrared light sources 11c and 11d in a state where the direction of the slit light is kept constant. The fundus reflection light of the slit light incident on the subject eye in a predetermined direction is projected onto the imaging element 21 via the front imaging optical system 10. Hereinafter, the slit-shaped image based on the fundus reflection light projected on the imaging element 21 is referred to as a “slit image”. The control unit 100 controls the imaging element 21 to image at least one frame, and detects the slit image based on the captured image. In the present example, at least the position of the slit image is detected.

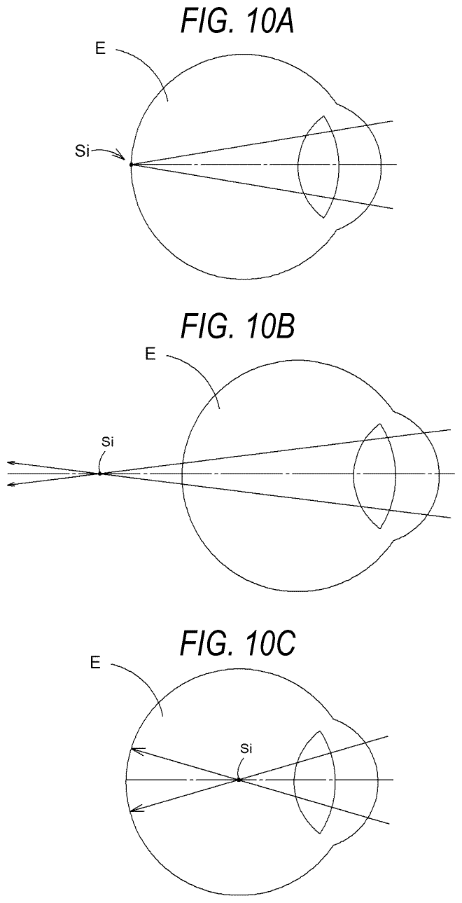

FIGS. 10A to 10C schematically illustrate light rays of the slit light for the subject eye. As described above, in the present example, in the anterior segment of the subject eye, images P1 and P2 (light projection regions P1 and P2) of the two light sources are formed axisymmetrically with respect to the optical axis L2. An opening image Si of the slit unit 12 is formed downstream of the image P1 and P2 of the light sources. The slit light is emitted to the fundus as two light rays from the images P1 and P2 of the two light sources toward the opening image Si.

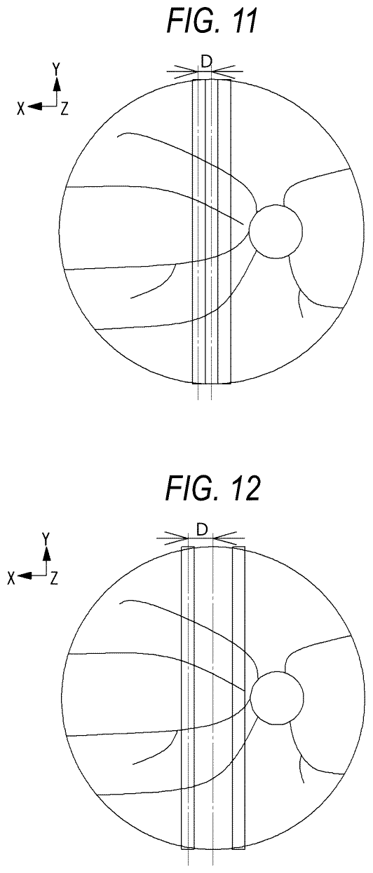

FIG. 10A illustrates light rays in a case where the direction of the slit light is kept constant and the slit unit 12 is arranged at the initial position (movement limit position on the minus diopter side). In this case, since the position of the opening image Si of the slit unit 12 is on the back side of the fundus of the subject eye E on the optical axis L2, the irradiation position of the slit light on the fundus is separated from the optical axis L2. As a result, as illustrated in FIG. 11, the slit image is detected at a position away from the optical axis L2 (the center of the image in FIGS. 11 and 12) on the imaging element 21.

For example, the control unit 100 moves the slit unit 12 from the initial position to the movement limit position on the opposite side, and acquires a distance D between the slit image and the optical axis L2 at each position. In this case, the light ray emitted to the fundus changes in the order from FIG. 10B to FIG. 10C. FIG. 10B illustrates light rays in a case where the direction of the slit light is kept constant and the diopter correction is appropriately performed. In this case, the position of the opening image Si of the slit unit 12 substantially coincides with the fundus. Therefore, the irradiation positions of the slit light on the fundus emitted from the images P1 and P2 of the two light sources coincide with each other as illustrated in FIG. 5. FIG. 10C illustrates light rays in a case where the direction of the slit light is kept constant and the slit unit 12 is arranged at the movement limit position on the plus diopter side. In this case, the position of the opening image Si of the slit unit 12 is on the front side of the fundus of the subject eye E on the optical axis L2. Also in this case, as illustrated in FIG. 12, the irradiation position of the slit light on the fundus is separated from the optical axis L2.

Therefore, the control unit 100 acquires the position of the slit unit 12 at which the distance D from the optical axis L2 to the slit image is minimized, and moves the slit unit 12 and the focusing lens 19 in the front imaging optical system 10 and the focusing lens 41 in the OCT optical system 30 to the position corresponding to the acquired position, thereby performing the focus adjustment. However, in this case, the correspondence relation between the slit unit 12 and the focusing lens 19 in the front imaging optical system 10 and the focusing lens 41 in the OCT optical system 30 is known.

In addition, fine adjustment of the focus may be performed in the OCT optical system 30. For example, the position of the focusing lens 41 may be adjusted so that the focus position matches a predetermined layer. The fine adjustment of the focus in the OCT optical system 30 is preferably performed at a stage where the adjustment of the OPL, the polarization, and the like is completed.

The diopter correction method in the present example is not necessarily limited thereto. For example, the distance D from the optical axis L2 to the slit image corresponds to the diopter of the subject eye. The appropriate positions of the optical elements (the slit unit 12, the focusing lens 19, and the focusing lens 41) for each diopter are known. Therefore, for example, the control unit 100 may acquire a diopter (an example of the focus evaluation value) of the subject eye E from the distance D from the optical axis L2 to the slit image at the initial position, and further perform focus adjustment by moving each optical element (the slit unit 12 and the focusing lens 19 in the front imaging optical system 10, and the focusing lens 41 in the OCT optical system 30) to an appropriate position corresponding to the diopter. The diopter may be acquired using a lookup table that stores in advance a correspondence relation between the diopter of the subject eye and the distance D from the optical axis L2 to the slit image at the initial position. The lookup table can be created, for example, by measuring the distance D from the optical axis L2 to the slit image at the initial position for a plurality of subject eyes or model eyes whose diopter is known.

### <OPL Adjustment>

Next, the control unit 100 adjusts the OPL and polarization in the OCT optical system 30.

In the present example, the optical path length adjustment is performed by changing the optical path length in the reference optical system. However, the present invention is not necessarily limited thereto, and the optical path length adjustment may be performed by changing the optical path length in the measurement optical system 40. The control unit 100 changes the optical path length stepwise by predetermined steps (for example, by about several mm in terms of air). The optical path length for acquiring the tomographic image of the fundus is specified based on an output signal output from the detector 33 in each step. After adjusting the optical path length so as to acquire the tomographic image of the fundus, the control unit 100 finely adjusts the OPL. For example, the optical path length may be adjusted such that the image of the fundus is arranged at a predetermined target position with respect to the imaging range in a depth direction. In the tomographic image of the fundus, a real image and a virtual image are generated at symmetrical positions with respect to zero delay. Since the real image and the virtual image can be appropriately formed, a predetermined one of the real image and the virtual image can be arranged at the target position.

### <Polarizer Adjustment>

In the present example, the control unit 100 drives the polarizer to adjust the polarization state between the measurement light and the reference light. When the polarization states of the measurement light and the reference light match, a stronger interference signal is obtained. Therefore, the polarizer is driven and controlled based on the signal intensity so that the signal intensity output from the detector 33 of the OCT optical system 30 is maximized.

### <OCT Image Capturing>

By receiving an operation input serving as a trigger for imaging, a scan for imaging is performed with the scan pattern determined in advance (step S6). The imaged OCT image may be stored in, for example, the storage unit 101. The imaged OCT image may be displayed on a screen (step S7). Further, a report may be generated based on the imaged OCT image.

### <Visibility of Fixation Target>

In the above description, it is desirable that the visual orientation of the examinee is appropriately guided with respect to the fixation target presented to the subject eye during the period from the alignment to the completion of the imaging of the OCT image. With respect to this, in the present example, since the point-shaped focus index is not projected, it is possible to appropriately guide the visual orientation of the examinee with respect to the fixation target and image the OCT image.

Further, in the present example, even if the wavelength band of the infrared light for fundus observation can be visually recognized by the subject eye, the fixation is hardly affected. First, the scanning with the slit light repeated between the alignment and the imaging of the OCT image is performed at a high speed, and from the subject eye, it appears as if the observation light is being emitted to the imaging range of the observation image at approximately the same time, making it difficult for the visual orientation to follow the moving slit light. Next, during the focus adjustment, the direction of the slit light is fixed to a certain direction in order to acquire the focus evaluation value, but since the imaging time of the slit image of one frame is generally sufficiently short (about several tens of milliseconds), there is no need to continuously emit the slit light for a long time in a state where the direction is kept constant in order to acquire the focus evaluation value. In this case, for example, during the focus adjustment, a slit image may be imaged with the direction of the slit light fixed at the time of acquiring the focus evaluation value, and scanning with the slit light may be performed to obtain the observation image for other periods except the time. In addition, while the fixation target is point-shaped and thus more likely to guide the visual orientation, the slit light is linear-shaped and is emitted to a relatively wider range, making it less likely to guide the visual orientation. As described above, according to the present example, it is possible to appropriately guide the visual orientation of the examinee with respect to the fixation target presented to the subject eye and image the OCT image.

Further, in the present example, when imaging the peripheral portion of the fundus, the visual orientation of the examinee is guided by the second fixation optical system 80, from a stage where the subject eye E is at a position farther than the appropriate operation distance WD2 with respect to the imaging unit 3, so that the visual orientation of the subject eye is smoothly guided with respect to the fixation target formed by the fixation light sources 71d to 71k of the fixation target unit 71. Therefore, it is possible to appropriately guide the visual orientation of the examinee without being affected by the slit light and image the OCT image.

In addition, since the visual orientation of the examinee is likely to be appropriately guided with respect to the fixation target, it is possible to reduce an intervention frequency of the examiner such as assisting the examination by the examiner from the start of the alignment to the completion of the imaging of the OCT image.

### <Modifications>

For example, in the above example, in order to acquire the focus evaluation value of the subject eye based on the slit image, the slit light is simultaneously emitted to the fundus from the two light projection regions P1 and P2 formed in the anterior segment of the subject eye. However, the present invention is not necessarily limited thereto. For example, the slit light may be selectively emitted from one of the two light projection regions P1 and P2. In this case, the focus evaluation value may be acquired based on the position of the slit image based on the selectively emitted slit light. In addition, slit images based on slit light alternately emitted from the two light projection regions P1 and P2 may be imaged as different frames. The focus evaluation value may be acquired based on the position of the slit image in each frame. When the fundus is selectively or alternately irradiated with the slit light from the two light projection regions P1 and P2, it is easy to specify the sign (plus or minus) of the diopter correction amount required in the optical system from the position of the slit image. Therefore, for example, it is not necessary to set the initial position of the slit unit 12 as the movement limit position, and it is easy to readjust the focus based on the slit image. Selectively or alternately irradiating the fundus with slit light from the two light projection regions P1 and P2 can be realized by turning on control of light sources 11c and 11d in the configuration of the above example. However, the present invention is not necessarily limited thereto, and light limiting members such as shutters that correspond to the light projection regions P1 and P2, respectively, and can be selectively opened and closed may be arranged on the optical path of the illumination light at a position conjugate to the anterior segment.

What is claimed is:

1. A fundus imaging device comprising:
a fixation optical system;
an OCT optical system;
an imaging optical system; and
a controller, wherein
the fixation optical system is configured to present a fixation target on a fundus of a subject eye,
the OCT optical system includes:
an OCT light source;
a split optical element configured to split light emitted from the OCT light source into measurement light and reference light;
a detector configured to detect a spectral interference signal between the measurement light reflected from the fundus of the subject eye and the reference light; and
a first focus adjustment unit configured to adjust a focus position of the measurement light with respect to the fundus of the subject eye,
the imaging optical system includes:
an irradiation optical system including a light source configured to emit infrared light as illumination light, and configured to form two light projection regions through which the illumination light passes on a pupil of the subject eye side by side in a first direction and irradiate the fundus of the subject eye with slit-shaped illumination light formed to be elongated along a second direction intersecting the first direction;
an optical scanner configured to deflect the illumination light in the first direction on the fundus; and
a light receiving optical system including a two-dimensional imaging element configured to form a light receiving region where fundus reflection light of the illumination light is extracted on the pupil of the subject eye so as to be sandwiched between the two light projection regions, and configured to receive the fundus reflection light of the illumination light, and
the controller is configured to:
acquire a focus evaluation value based on a slit image formed by the slit-shaped illumination light on the two-dimensional imaging element of the imaging optical system in a case where the optical scanner deflects in a predetermined direction; and
perform diopter correction control of the OCT optical system by driving the first focus adjustment unit of the OCT optical system based on the focus evaluation value.

2. The fundus imaging device according to claim 1, wherein
the OCT light source is configured to emit low-coherence light having a center wavelength included in a range of 820 nm to 880 nm, and
the detector is a spectrometer.

3. The fundus imaging device according to claim 1, wherein
the controller is configured to fix a deflection direction of the optical scanner to image the slit image at a time of acquiring the focus evaluation value, and is configured to drive the optical scanner to perform scanning with the illumination light for other periods except the time to acquire an observation image based on a light receiving signal obtained from the two-dimensional imaging element.

4. The fundus imaging device according to claim 1, wherein
the fixation optical system is configured to share a part of an optical system with the OCT optical system and the imaging optical system, and is configured to present a peripheral fixation target for arranging an optic nerve head of the subject eye in a central portion of an imaging range,
the fundus imaging device further comprises a fixation light source that is arranged around an objective lens in the OCT optical system and the imaging optical system, and is configured to present an external fixation target at a position corresponding to the peripheral fixation target, and
the external fixation target guides a visual orientation of an examinee to a direction corresponding to the peripheral fixation target, from a stage where the subject eye is at a position farther than an appropriate operation distance.
